(19) United States
(12) Patent Application Publication
(10) Pub. No.: US 2009/0318798 A1
(43) Pub. Date: Dec. 24, 2009

(54) FLEXIBLE VISUALLY DIRECTED MEDICAL INTUBATION INSTRUMENT AND METHOD

(76) Inventors: Errol Singh, Columbus, OH (US); Allen Stock, Gahanna, OH (US); Walter Russell, Gahanna, OH (US)

Correspondence Address: GALLAGHER & DAWSEY CO., L.P.A., P.O. BOX 785, COLUMBUS, OH, 43216, US

(21) Appl. No.: 12/488,940

(22) Filed: Jun. 22, 2009

Related U.S. Application Data
(63) Continuation-in-part of application No. 12/214,944, filed on Jun. 23, 2008.

(51) Int. Cl. A61B 6/12; A61M 25/00

(52) U.S. Cl. 600/424; 604/544

(57) ABSTRACT

A flexible medical intubation instrument provided for placement into an animal or human patient comprises a catheter with at least one longitudinally extending lumen or channel. A fixed or slideably removable sensor cable is at least partially contained within the channel, having a sensor for sensing a characteristic or condition. While enabling observations through the sensor channel, the working channel may simultaneously function as a drain or an irrigation duct, a feeding tube, or to provide a passage for the insertion of one or a succession of surgical devices such that the catheter serves as a protective artificial tract or liner as surgical devices are inserted and removed through it in succession so as to minimize tissue trauma.

### CROSS-REFERENCE TO RELATED APPLICATIONS

This application is a continuation-in-part application of, and claims priority to, U.S. patent application Ser. No. 12/214,944, filed Jun. 23, 2008.

### STATEMENT REGARDING FEDERALLY SPONSORED RESEARCH OR DEVELOPMENT

Not Applicable.

### FIELD OF THE INVENTION

This invention relates to medical instrumentation and more particularly to a method and apparatus for facilitating intubation of an animal or human patient.

### BACKGROUND OF THE INVENTION

In many medical procedures it is often necessary to place an instrument into the body of the patient for drainage, for viewing a part of the body, or for performing a surgical operation such as the endoscopic removal of a tumor, to take a biopsy, or for feeding the patient. The invention may have general application in medicine including the field of urology as well as in the field of gastroenterology and in other medical and surgical specialties. The placement of a catheter in the urethra for the purpose of draining urine or for diagnostic purposes, for example, is one of the most common urological procedures for draining urine or fluid to determine the amount of urine present, to diagnose problems, or to maintain anatomic continuity. This procedure is commonly performed by inserting the catheter manually while noting any resistance to forward movement as shown by a failure of the catheter to slide smoothly into the urethra. While most placements proceed without problems, typically more than forty percent of male urinary catheter placements are difficult because of the problematic normal anatomy of the male lower urinary tract such as the external sphincter, the S-curve of the bulbous urethra and angulated prostatic urethra and/or pathologic conditions, such as urethral stricture disease, stones, trauma, tumors, enlarged prostate, iatrogenic false passages, and/or congenital disorders causing a substantial burden on the delivery of effective care through the healthcare system. The most common problem is tetany, a spasm of the external urinary sphincter or stricture of the urethra. Stones, and even clots descending from the bladder, also constitute urethral obstructions. In addition, urethral lumen calibers vary considerably, and particularly with urethritis, BPH, urethritis stricture disease and prostate disorders in males. These costs to the healthcare system, hospitals, clinics and doctors' offices are substantial. In addition, the delay in servicing urological catheter patients in a timely manner constitutes poor medical efficiency, delivery, and control. When difficulty is encountered, the resulting frustration among healthcare professionals, especially nurses, physician extenders and physician assistants, creates a very real feeling of ineffectiveness on the part of these healthcare workers, to say nothing of the dissatisfaction on the part of the patients caused by the delay and added discomfort. Difficult catheterizations can also be a source of urinary tract infection subsequent to damage of the urinary epithelium. While the dollar cost to the healthcare system is not the only concern, such elements as added labor and material costs, time delays for patient rectification, excess space and equipment required, catheter kit value, nurse technician and physician costs constitute an expense to the healthcare system of surprising proportions. The best available current data indicates about 55,000 urinary catheter placements are made in the United States per day. Of these, conservatively about 40% are difficult which means that they require multiple advances and pull-backs of the urinary catheter to negotiate the urethra, multiple catheters on the same patient, several staff workers attending to the same patient, or special instrumentation such as filoforms/followers, cystoscope or radiologic services.

Two prior U.S. patents by the present inventor; U.S. Pat. Nos. 6,599,237 and 6,994,667 are directed to some of these problems An important consideration is the high cost of surgical instruments, which may be from several hundred to several thousand dollars. Some endoscopes for example may cost more than $10,000.00. Other instruments may be suited for urological use but not be suited for use in gastroenterology. Certain intubation devices such as the Councill catheter are only capable of a blind insertion and must rely on a guide wire to navigate to the bladder. Consequently, if the Councill catheter encounters resistance during insertion, there is no way to know its cause. By contrast, one aspect of the present invention is the provision of a visually directed instrument to permit continuous observation of the field just ahead of the tip of the instrument during insertion so that abnormal conditions such as obstructions or other anomalies can be continuously observed and dealt with by the clinician as the instrument is being inserted. Currently, in the field of gastroenterology, intubation by means of a nasogastric tube is commonly carried out blindly or by means of a wire guide for placement into the stomach. Any obstructions, anomalous conditions, or anatomical idiosyncrasies can interfere with successful insertion of the tube. Heretofore irrigation has required an endoscope with a passage for irrigation. Moreover, no provision is made for sensing conditions at or near the distal tip of the intubation instrument with traditional analog sensors and/or actuators or smart digital sensors or actuators.

It is therefore one object of the present invention to provide surgical instrumentation for intubation that provides a sensor or multiple sensors including chemical, ultrasound, pressure, temperature sensors, or a visual sensor such as a highly versatile visually directing sensor to facilitate insertion of a catheter or other tube into the body of an animal or human patient.

Another object of the invention is the provision of a surgical instrument for visually directed intubation that is suited for use in the field of urology as well as in gastroenterology and other surgical specialties.

Yet another object is to provide a surgical intubation instrument for providing visually directed placement into the body of the patient that makes possible a dramatic reduction in the cost of the instrument.

Another object is to provide a way of permitting a medical procedure to be conducted through a catheter to protect the patient from injuries while observing a selected part of the body of the patient.

A more specific object of the invention is the provision of an improved surgical intubation instrument that allows a catheter to be routinely passed even in a difficult situation, includes a provision for enabling the patient to tolerate the catheter more readily by reducing pain and the risk of injury or infection, the elimination of many steps and procedures currently used to pass a common Foley style catheter, as well as the need for a guide wire or a filoform/follower procedure or the need for cystoscopy to pass a guide wire that is thereafter used for directing the movement of a catheter so as to reduce the frequency of complications during the insertion of a catheter.

A further object is to provide the forgoing characteristics and advantages while permitting the insertion of surgical instruments into the body without the need to remove a previously inserted catheter as well as to permit the passage of relatively large surgical instruments that cannot be inserted through an ordinary catheter.

These and other more detailed and specific objects of the invention will be better understood by reference to the following Figures and detailed description which illustrate by way of example of but a few of the various forms of the invention within the scope of the appended claims.

### SUMMARY OF THE INVENTION

The present invention provides a method and apparatus for facilitating medical intubation procedures. In accordance with one aspect of the invention, there may be provided a flexible direct vision viewing instrument or viewer that includes a catheter or sheath formed from a highly flexible biocompatible polymer such as natural or synthetic rubber or plastic having a longitudinal working channel extending the length of the catheter with an outlet port that may be positioned in alignment with the channel at the distal end of the catheter. The catheter may have a second longitudinal channel or lumen that contains a flexible sensor cable such as viewing cable for optical sensing. In place of or in conjunction with an optical sensor, there can be provided any of various kinds of sensors such as a chemical sensor, a pH sensor, a temperature sensor, in vivo infection, or the like. In the case of a visual sensor, one of the channels contains an optical cable providing illumination in the proximity of the distal end of the catheter for enabling the body of the patient to be viewed during placement of the instrument through a body opening or percutaneously through a surgical opening. An objective optical sensor or other sensor at the distal end of the cable provides information, e.g. continuous viewing the body of the patient just ahead of the tip of the instrument during insertion of the instrument as well as after placement of the instrument within the body. The invention may be adapted to be produced in either a disposable version or a reusable version that can be sterilized after use.

The invention also provides a catheter that may be able to serve as a working sheath which can be thought of as a temporary and removable artificial tract or liner that is placed through an opening in the body of the patient at the beginning of a surgical procedure to facilitate endoscopic evaluation and treatment of the digestive tract, urinary tract, or other body cavity while minimizing trauma and patient pain. During use, it allows multiple insertions and removals, i.e., the interchange of endoscopic instruments, catheters, sensors, drains, etc. The viewing cable can act as a stiffener during insertion into the patient to provide a greater degree of firmness, especially when the sheath or catheter is relatively thin or tends to fold back upon itself during insertion. Once in place, the viewing cable can be removed and replaced by other sensors such as a temperature sensor, a pH sensor, or an infection sensor, or by other medical devices. At its proximal, i.e. exterior end, the lumen of the sheath may have an entry port for instruments with a removable cap that provides a nipple seal to preclude backflow of fluid from the body after the visual element or other sensor has been removed. The instrument can be placed into the stomach or other part of the digestive tract or the urethra under direct vision, i.e., with a flexible condition sensor extending through the sheath to act as a temperature, pH, or visual sensor. The sensor can include a sensor/actuator cable that provides an interoperable medium for transmitting optical or electrical signals, e.g. a fiber-optic bundle for illuminating and viewing a body cavity through the sheath, both during the insertion of the sheath and thereafter.

In some embodiments, the instrument may have catheter with a single longitudinal channel, and the sensor may occupy only a small portion of the longitudinal channel. In other embodiments, a catheter and sensor may have slightly differing working lengths, to help maintain a predetermined spatial arrangement between the catheter and sensor during stretching or compression of one of both of these elements. In yet another embodiment, a catheter wall thickness may vary in conjunction with a longitudinal channel diameter in order to maintain a constant over diameter of the catheter.

### THE FIGURES

FIG. 1 is a side elevation view of one embodiment of the invention showing a viewing device at its proximal end;

FIG. 2 is a longitudinal vertical sectional view of an embodiment of the instrument of FIG. 1 on a larger scale;

FIG. 3 is a vertical transverse sectional view taken on line 3-3 of FIG. 2;

FIG. 4 is a vertical transverse sectional view taken on line 4-4 of FIG. 2;

FIG. 5 is an end view taken on line 5-5 of FIG. 2;

FIG. 5A is a partial enlarged vertical sectional view of the distal end of an embodiment of the instrument shown in FIG. 2 on a larger scale;

FIG. 5B is a partial enlarged vertical sectional view of the distal end of an embodiment of the instrument showing an objective lens built into the end of the catheter;

FIG. 6 is a side elevation view partly in vertical section showing an embodiment of the instrument of the invention in place within the male urethra;

FIG. 7 is a partial front elevation view of a patient showing a medical intubation appliance of an embodiment of the present invention connected to the patient for gastric feeding;

FIG. 8 is a side elevation view partly in vertical section to show an embodiment of the invention in use as a nasogastric tube;

FIG. 9 is a vertical longitudinal sectional view of an embodiment of the tube of FIG. 8 on a larger scale;

FIG. 10 is a transverse vertical sectional view taken on line 10-10 of FIG. 9;

FIG. 11 is an end elevation view taken on line 11-11 of FIG. 9;

FIG. 12 is a transverse sectional view showing an optional expansion feature in accordance with an embodiment of the invention as it appears prior to use;

FIG. 13 is a transverse vertical sectional view of FIG. 12 as it appears after being dilated by the insertion of an oversized surgical device through its central lumen;

FIG. 14 is a schematic diagram of an embodiment of the viewing instrument and camera assembly;

FIG. 15 is an elevated perspective view of an embodiment of the catheter of the instant invention;

FIG. 16 is an elevation view of the catheter of FIG. 15;

FIG. 17 is an end view of the proximal end of the catheter of FIG. 15;

FIG. 18 is an end view of the distal end of the catheter of FIG. 15;

FIG. 19 is a longitudinal section view of the catheter of FIG. 15;

FIG. 20 is an elevation view, including detail, of an embodiment of the sensor of the instant invention;

FIG. 21 is a longitudinal section detail view of the distal end of a catheter and sensor of an embodiment of the instant invention, showing the sensor partially retracted within the catheter;

FIG. 22 is a longitudinal section detail view of the distal end of a catheter and sensor of an embodiment of the instant invention, showing the sensor and catheter in a working intubation position;

FIG. 23 is a longitudinal section view of the distal end of a catheter and sensor of another embodiment of the instant invention in a working intubation position;

FIG. 24 is a longitudinal section view of the distal end of a catheter and sensor of FIG. 23 with the catheter under longitudinal axial compression;

FIG. 25 is a longitudinal section view of a catheter and sensor of an embodiment of the instant invention in a working intubation position;

FIG. 26 is a longitudinal section schematic illustration showing the distal ends of a hypothetical sensor and catheter in an approximate working position;

FIG. 27 is a longitudinal section schematic illustration showing hypothetically the distal end of a sensor extended beyond the distal end of a catheter;

FIG. 28 is a longitudinal section schematic illustration showing hypothetically the distal end of a sensor retracted within the distal end of a catheter; and

FIG. 29 is a longitudinal section view of the distal end of another embodiment of a catheter.

### DETAILED DESCRIPTION OF THE PREFERRED EMBODIMENTS

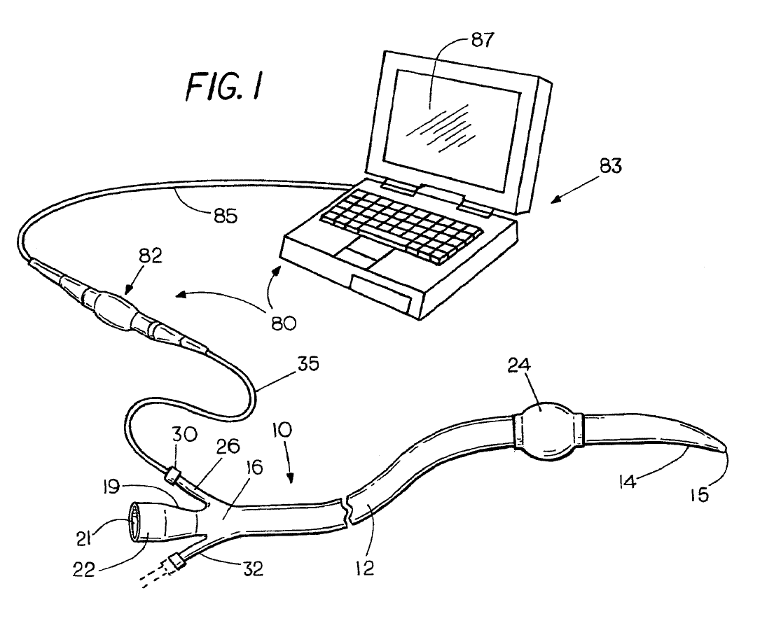

Refer now to the FIGS. 1-29 wherein the same numerals refer to corresponding parts in the several views. The invention will be described by way of examples, all of which are intended for illustration only and not for limitation, which illustrate a visually directed intubation instrument in accordance with the invention that can be placed into the body of the patient under direct and continuous visual control in any of a variety of different surgical specialties. The invention is especially versatile and can be dimensioned and configured for use in urology, in gastroenterology, and in other surgical fields. The various embodiments illustrate the versatility of the invention since it can be employed as a drain or for exploratory purposes as well as a working channel to be used during a surgical operation or even in the field of gastroenterology as a feeding tube.

In certain embodiments, the instrument 10 comprises a flexible catheter 12 formed from natural or synthetic rubber or from a flexible biocompatible polymer of any suitable known composition such as synthetic rubber, latex rubber, polytetraflouroethylene (PTFE), polyethylene (PE), perfluoroalkoxy (PFA), polyurethane (PU), perfloromethylvinylether (IVrFA), perfluoropropylvinylether (PPVE) or other polymeric materials which would be apparent to those skilled in the art. The flexibility of the catheter 12 is apparent in FIG. 1. The catheter can also be thought of as a sheath since it is able to function in some instances as a protective sleeve for accommodating other surgical instruments that are passed through it as will be described more fully below. The catheter 12 may have a proximal end 16 and a distal end 14 terminating at a tip 15. Inside the catheter 12 is a lumen that serves as working channel 18 which extends the entire length of the catheter 12 and may be provided with a distal opening 18a at one end and a proximal opening 22 at the opposite end. It will be noted that the distal end 14 portion of the catheter 12 adjacent the opening 18a may be tapered so that its outer diameter may be progressively reduced proceeding toward the opening 18a at its tip 15. The catheter 12 can vary in length to suit the application to which it is applied, but may be typically from about 30 cm to 50 cm in length and may be preferably about 40 cm in length when it is to be used in gynecological procedures. It can be longer, say, 50 cm in length, when used in the male, for example, in a transurethral resection of a bladder tumor. For transurethral use, the outside diameter may be typically about 9 mm (27 French) and the inside diameter about 5 mm (15 French). It should be understood that the dimensions presented herein are merely typical and can be varied to suit the circumstances in which the instrument is used. When used as a nasogastric or jejunostomy tube it can be at least 100 cm or more in length.

At the distal end 14 of the catheter 12 may be provided an inflatable circumferentially extending annular balloon 24 formed from a ring of resilient elastomeric biocompatible material that extends around the catheter 12 adjacent the distal opening 18a. Inflation air or liquid may be supplied to the balloon 24 when required through a tubular extension 32 at the proximal end 16 of the catheter 12 which communicates through inflation duct 33 through channel 28 with the balloon 24. If the catheter 12 is formed from an elastomer such as rubber, the balloon 24 can be integral with the sheath. However, if the catheter 12 is formed from a firm plastic material such as polypropylene, the balloon 24 may be formed from rubber that may be bonded to the outside surface of the catheter 12 by means of a suitable adhesive. The free end of the tubular extension 32 may be provided with an inflation port through which inflation fluid (gas or liquid) can be introduced and retained until a valve, e.g. Luer lock 31 is opened.

It will be noted that the catheter 12 may be provided with three channels or lumens including a lateral channel 34 that serves to accommodate the visual element, in this case a flexible fiber-optic bundle 35 for illumination and viewing, a channel 18 that can be used for drainage or as a working channel to accommodate rigid or flexible instruments that are passed through it in succession during a surgical operation, and the inflation channel 28 already described for inflating the balloon 24. It will be noted that the proximal end of the working channel 18 may have an enlarged partially tapered entry port 19 with an enlarged circular open mouth 21 to give the distal end of the working channel 18 a funnel-like entry passage to accommodate the insertion of instruments during the course of a surgical operation. If desired, as shown in FIG. 3, the catheter 12 can be provided with an additional optional longitudinally extending duct 25 having an inlet port at the proximal end of the catheter and an outlet port 27 positioned at the proximal end of the balloon 24 as described in U.S. Pat. Nos. 6,599,237 and 6,994,667 for the purpose of introducing topical anesthetics and other medicament into the passage through which the catheter has been inserted where it will be trapped between the catheter and the surrounding body tissue.

For some purposes, the fiber-optic bundle 35 may be embedded within the lumen 34 of the catheter 12 so as to be fixed in place and thus not removable during the course of its useful life. However, the fiber-optic bundle can, if desired, be made removable in certain applications, for example, when the lumen 34 is used for a lavage and the central lumen 18 used for drainage. An embedded optic bundle provides a very effective yet inexpensive flexible visual catheter that can be sterilized and used repeatedly or can even be produced in a disposable form because of its low cost. This is an important feature since sterilization is expensive and sometimes may not be completely effective.

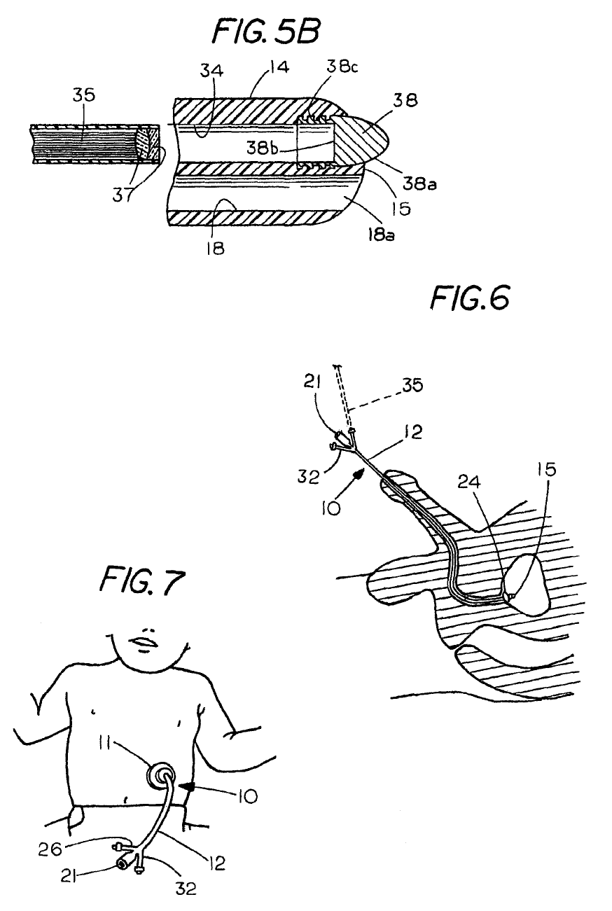

As best seen in FIG. 5A, the fiber-optic bundle 35 may be provided with a viewer comprising an objective viewing element, e.g. a lens 37 that may be adjacent to the opening 18a of the working channel 18. It will thus be seen that the both the objective viewing element 37 of the fiber-optic bundle 35 and the outlet port 18a of the working channel 18 face forwardly along laterally spaced apart parallel axes 39 and 41 (FIG. 5A) of which axis 39 may be the optic axis of lens 37. The objective viewing element comprising the lens 37 in the embodiment illustrated projects in this case slightly beyond the free end or tip 15 of the catheter which makes wide angle viewing possible. However, if desired, for certain applications, the lens 37 can be recessed slightly within the lumen 34 so that it does not extend beyond the tip 15 of the catheter 12, but in that event wide angle viewing will be severely limited or impossible. The location of the port 18a on the end of the catheter rather than on its side allows channel 18 to be used for irrigation and other applications without the need of an endoscope for that purpose. Thus, the invention enables expensive endoscopes to be dispensed within many instances.

Refer now to FIG. 5B which shows a modified form of catheter 12 in which the lumen 34 at the tip 15 of the catheter 12 may be provided with a built in, i.e. permanently attached, objective lens 38. In the example illustrated, lens 38 may have a convex outer surface 38a to assure smooth passage through the urethra or other body opening and a planar inner surface 38b. The lens surfaces can, however, have any desired configuration to provide the desired optical qualities as will be apparent to those skilled in the art. To provide a secure connection, the lens 38 can be provided with an externally ribbed tubular bonding sleeve 38a adhered to the inner wall of the lumen 34 to act as a non-removable connection. One or more lenses 37 at the distal end of the optic cable 35 may be selected to complement lens 38 so as to reduce or eliminate chromatic, spherical, or fisheye aberration or other possible aberration to thereby provide an integrated lens combination when the cable 35 may be inserted to bring the lens 37 at its end into contact with the surface 38b of lens 38. However, if lens 38 alone provides a good image, the lenses 37 can be eliminated and the ends of the optic fibers themselves brought into contact with the lens 38 when the optic cable 35 may be inserted. The lens 38 can thus provide a smoothly contoured external surface outside of and ahead of the tip 15 for achieving excellent wide angle viewing while at the same time being shaped to assure easy movement through restrictions or around obstructions. In addition, lens 38 may be permanently positioned in the optimum location at the end of the tip 15 while sealing the lumen 34 to prevent the entry of fluid or other foreign material.

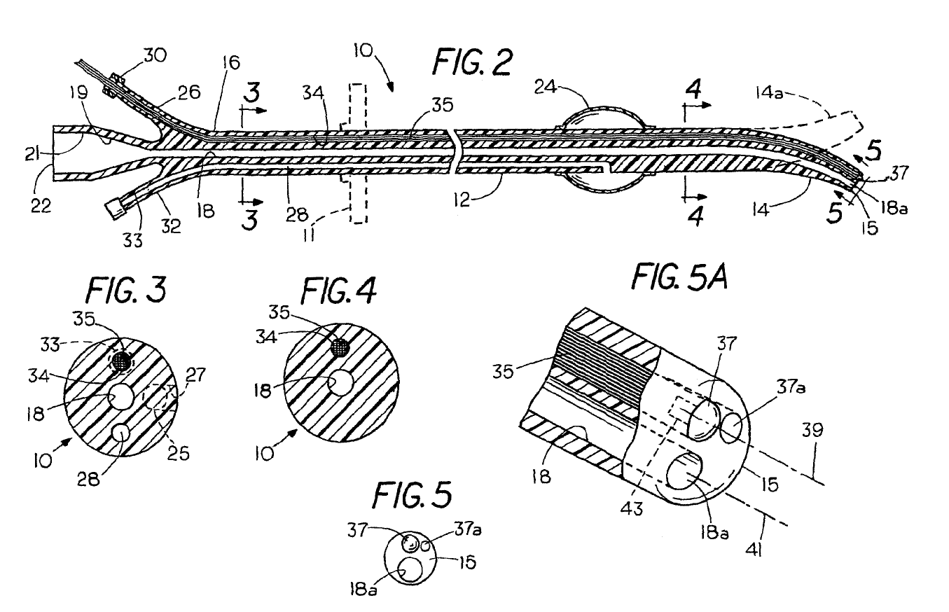

Upon encountering an obstruction during insertion, the curve shown in the tip 14 can be redirected by the operator for steering the catheter to facilitate insertion, i.e. by passive steering. The flexibility of the entire catheter including the distal end 14 is shown in FIG. 1 as well as at 14a in FIG. 2 which illustrates how the curved tip 14 can be deflected in any direction. Thus, 14a represents an alternate position of the tip as it appears when deflected upwardly or in any other direction, a feature made possible owing to the flexibility of the composite structure composed of the catheter 12 itself and the flexible visual element or cable 35.

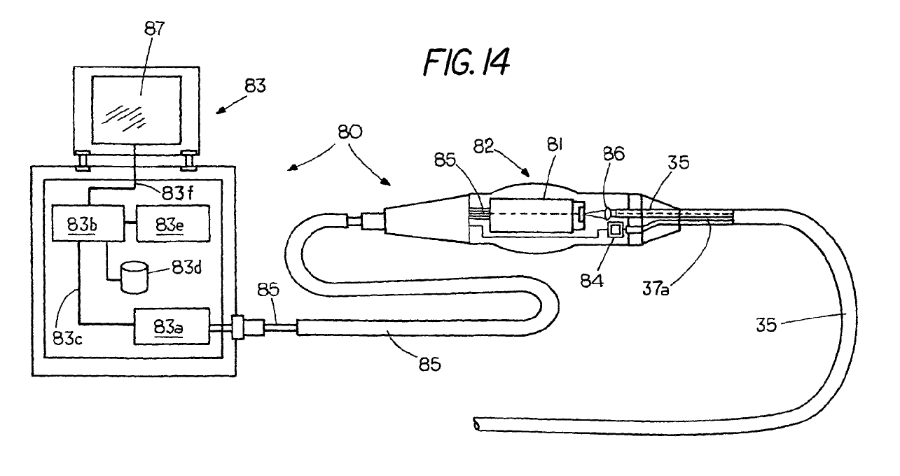

The flexible fiber-optic cable 35 which has been shown diagrammatically, can consist of crystal or glass and/or polymeric optical fibers of any suitable commercially available construction for illumination and viewing. In one preferred form, the fiber-optic bundle 35 may have a fiber bundle terminating at 37a (FIG. 5A) for providing illumination from a light source 84 (FIG. 14) and a second set of fibers coupled to the lens 37 for carrying an image to a viewer or other output device 80 (FIG. 1). When cable 35 is removable, it will be seen that both the illumination fibers and imaging fibers are contained together in one removable bundle. In an alternative form, the optical fibers can be replaced by electrical conductors connected to another type of sensor such as an electronic microcamera 43 (FIG. 5A). While the medium for transmitting the optical representation of the object viewed at the tip of the catheter can be a flexible fiber optic cable made of glass or a polymeric material, when microcamera 43 is used, the medium may be a flexible conductive wire consisting of copper or a pure element such as gold or silver or an alloy formulated to meet resistivity requirements, or a conductive or non-conductive liquid. For wireless transmission of video signals by radio frequency signal transmission from microcamera 43 the medium can be a pure gas or mixture of gasses or vacuum. The instrument 10 may be thus provided with a sensor such as the objective viewing lens 37 focusing an image onto the microcamera 43, for example, a suitable commercially available integrated circuit having light sensitive material onto which an image is focused such as a model FSC2 camera by Schoelly GmbH of Denzlinger, Germany.

Any of several types of actuators or sensors can be used for determining the state of one or more characteristics or conditions in the region ahead of or surrounding the sensor. The term “sensor” or “condition sensor” herein includes any of the following: a visual sensor, i.e. an optical viewer for producing an image, a chemical sensor including O₂, CO₂, and pH sensors, infection sensor, a pressure sensor, an audio or sonic sensor, or a temperature sensor among others. Moreover, the sensor can be a multi-sensor device which measures multiple phenomena simultaneously in real-time thus avoiding the removal of one sensor and the insertion of another sensor. For example, the sensor may incorporate at least one sensor bus. A sensor bus is a networking interface for sensors (data inputs) and actuators (data outputs). The many advantages for this type of device inter-connection technology include: reduced wiring and wire harness costs, support for intelligent devices, promotion of sensor and actuator interoperability, improved system diagnostics and mean time to repair, and support for peer-to-peer or distributed control.

Each sensor may be connected to an appropriate output device 80 (FIG. 1). The output device can be a meter or oscilloscope, video display unit, or other suitable output device well known to those skilled in the art. The removal of one sensor such as an optical sensor cable following insertion, allows replacement with a different kind of sensor such as a chemical sensor or temperature sensor which may be then inserted into the catheter through lumen 34 while the catheter 12 remains in place as a protective sheath within the body of the patient while sensing one or a series of different conditions or characteristics in the region ahead of or surrounding the sensor. The sensor cable can also transmit actuator signals to a proximal output instrument 83 (FIGS. 1 and 14), e.g. actuator signals for performing a predetermined function such as actuating a signal light or audible alarm (not shown) when the temperature or pH exceeds a critical level or to turn on a visual display screen, etc. The actuator can also be a valve for metering medication or anesthetic to the body tissue.

In the embodiment shown in FIGS. 1 and 14 by way of example, the fiber-optics 35 comprising glass or polymeric fibers exit the catheter at 30 to an output device 80 such as a viewing instrument which in this case is a light source and camera assembly 82 provided to receive an image from the objective lens 37 and a portable display monitor 83. The camera assembly 82 includes a miniature electronic integrated circuit camera 81 as well as a light source, e.g. the LED 84 for illuminating the area ahead of the lens 37 via fiber bundle 37a. The camera 81 may be connected by electrical bus 85 to the display monitor 83 which includes a video display screen 87 for displaying the image received from the objective viewing lens 37. During operation, the image from optic cable 35 may be focused by lens 86 onto electronic camera 81. While various known data display processor circuits can be employed, the display monitor 83 in this embodiment includes camera and light interface 83a feeding data to a system control 83b via bus 83c which may be coupled to data storage unit 83d and to a data acquisition and processing center 83e. The system control 83b feeds data to LCD color monitor 87 via bus system 83f in video format for displaying an image of the patient's body. One suitable electronic camera and light source assembly 82 is FSC2 (FlexiScope 2). The signal from bus 85 can also be routed to digital output ports (not shown) to display the image on a local color monitor or for streaming the video over the Internet. If the signal is to be stored for future use, the video signal may be processed through a computer hard drive for storage. The invention makes possible the continuous display of the image of the body passage obtained from lens 37 in real time as the catheter 12 is being inserted so that any discontinuities or obstructions can be observed and circumvented during the insertion procedure. Following insertion, an image of the urinary tract, gastrointestinal tract, or other body cavity that has been entered can be observed. If a sensor other than an optical sensor is used, the condition being sensed, e.g. the temperature, chemical composition, pH, etc. at the distal tip 15 of the instrument can be monitored on a suitable output device, e.g. meter or oscilloscope, etc. that may be used in place of the display monitor 83. If desired, the microcamera 43 (FIG. 5A) can also include a radio signal transmitter for transmitting a signal depicting or representing a condition or a visual image, in which case the radio transmission sent to the output device replaces the electrical bus 85 and fiber-optics 35 which are then eliminated. The cable 35 may be “resposable” after use, i.e. it can be pulled out of the catheter 12, cleaned and resterilized, inspected for functionality, then inserted into a new and sterile catheter. It may be then inspected to determine that it is functioning properly and is ready for its next use. The catheter 12 may be intended to be disposed of after each use. After a specified number of uses, the cable 35 may be also disposed of The cable 35 is preferably compatible with standard sterilization techniques such as EtO (ethylene oxide), glutaraldehyde, Steris, Sterrad sterilization or other industry standard sterilization techniques.

The transmission cable 35 as already mentioned can also be embedded in the catheter 12. The term “embedded” or “non-removable” herein is intended to mean that the cable 35 whether it be fiber-optics or an electrical cable is mounted securely enough so that it is not meant to be removed or easily removed in a simple manner by the user, although it is apparent, however, that it might be possible for a person to remove even an embedded cable with sufficient time and effort. The embedded cable can be held in place either mechanically, for example by means of surface irregularities which are gripped by the surrounding rubber of the catheter 12, or by being bonded in place within the passage 34, i.e. held in place by adhesion as the rubber or other flexible polymer forming the catheter 12 is cured. During manufacture, a cable 35 can be inserted into the passage 34 after the catheter has been completely formed then bonded in place or, if desired, it can be molded in situ as the catheter is being molded and before the polymer is cured or otherwise fixed within the catheter in any other manner known to those skilled in the art.

It is important to note that both the cable 35 and the catheter 12 are highly flexible so that together they form a composite structure which can flex in any direction as it is being inserted. This may be especially advantageous during a difficult passage or through a curved duct such as the male urethra or when an obstruction may be encountered. Flexing of the entire catheter is illustrated in FIG. 1. Flexing of the distal tip 14, e.g. to alternate position 14a is shown in FIG. 2 so as to enable the instrument to bend around corners or dodge obstructions. The cable 35 can also add a degree of stiffness to the instrument 10 so that sufficient stiffness may be provided to ensure that the entire instrument consisting of the catheter 12 and cable 35 can be easily inserted even through a tight passage, e.g. through the urethra without buckling, a problem sometimes referred to as a “wet noodle” effect wherein the entire instrument buckles as an axial force is applied to the proximal end by the operator in an attempt to push the distal end around a curve, past an obstruction or under other circumstances where resistance may be encountered. If desired, to provide additional stiffness, the cable 35 can be enclosed in a tubular casing 33 (FIG. 3) enabling it to serve as an obturator having a predetermined stiffness which makes the instrument 10 less subject to the possibility of buckling when axial pressure is applied.

Refer now to FIG. 6 which illustrates how the catheter 12 may be inserted into the male urinary tract to allow examination of the urethra and the bladder. It will be noted that the catheter 12 may be able to easily flex so as to negotiate curves in the urethra without difficulty and as the instrument is being inserted, the image just ahead of the distal end of the instrument can be continuously observed while noting pathological conditions or abnormalities in case the insertion becomes difficult or an obstruction may be encountered. If the optical cable 35 is embedded, i.e. fixed in the catheter 12, it remains in place following insertion thereby making continuous observation possible. The working channel 18 which can be temporarily plugged by means of a cap or other seal (not shown) may be then opened at its proximal end to allow one or several successive instruments to be introduced through the open mouth 21 as required during a surgical operation by passing them through the channel 18 into the bladder or other organ while the catheter 12 remains in place, thereby serving as a protective sheath in the manner described in U.S. Pat. Nos. 6,599,237 and 6,994,667 to prevent injury to the patient. The present invention however, may have the added benefit of permitting visual observations to be made continuously via the optic cable 35 while the working channel 18 (FIG. 2) may be used contemporaneously for drainage, for the passage of instruments used in surgery, or for any other purpose.

An important feature of the invention is an ability of any channel (channel 18 or 34) to be used for irrigation of the bladder or other organ, whereas heretofore an endoscope was required for this purpose. The invention, besides providing visualization, thus allows irrigation to be performed without the need for an expensive endoscope. Once the instrument 10 has been completely inserted, the balloon 24 may be inflated by introducing a fluid or gas through the passage 28 to hold the catheter 12 in place.

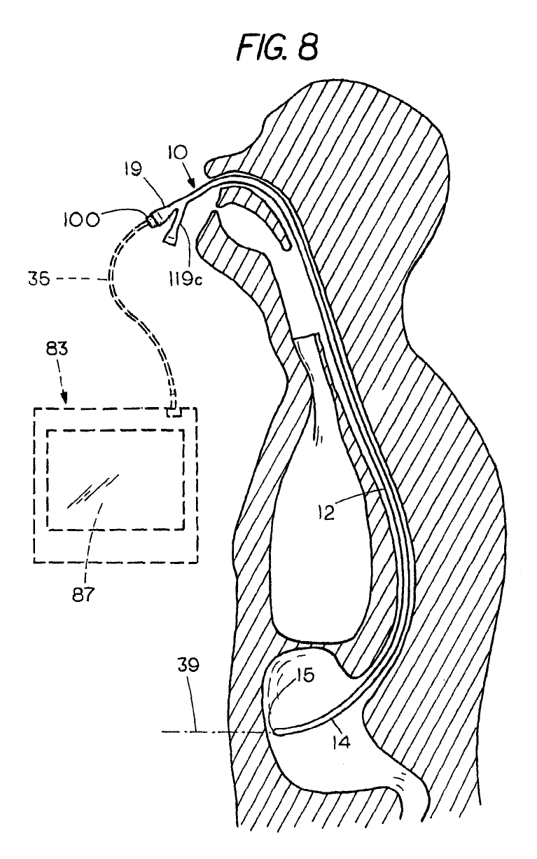

Refer now to FIG. 7 which illustrates how the invention can be used in gastroenterology, in this case as a gastronomy/gastrostomy tube that serves as a gastric feeding tube. When used as a gastric feeding tube, the instrument 10 may be preferably provided with an abdominal mounting disc 11 which may be bonded conventionally to the outside wall of the abdomen to hold the instrument which is inserted percutaneously in place where it enters the abdomen through the skin. The tube 10, which can be referred to as a percutaneous endoscopic gastrostomy tube, provides a convenient visually directed access route for the delivery of long-term enteral nutrition through the stomach. It may be surgically placed in the abdominal wall as shown in FIG. 7 below the rib cage and slightly to the left in this case for feeding an infant. The optic cable 35 or other condition sensor permits continuous visual or non-visual monitoring both during insertion and following insertion. When used as a feeding tube as shown in FIG. 7, the catheter 12 may be held in place by means of the inflated balloon 24 as well as sutures, if desired.

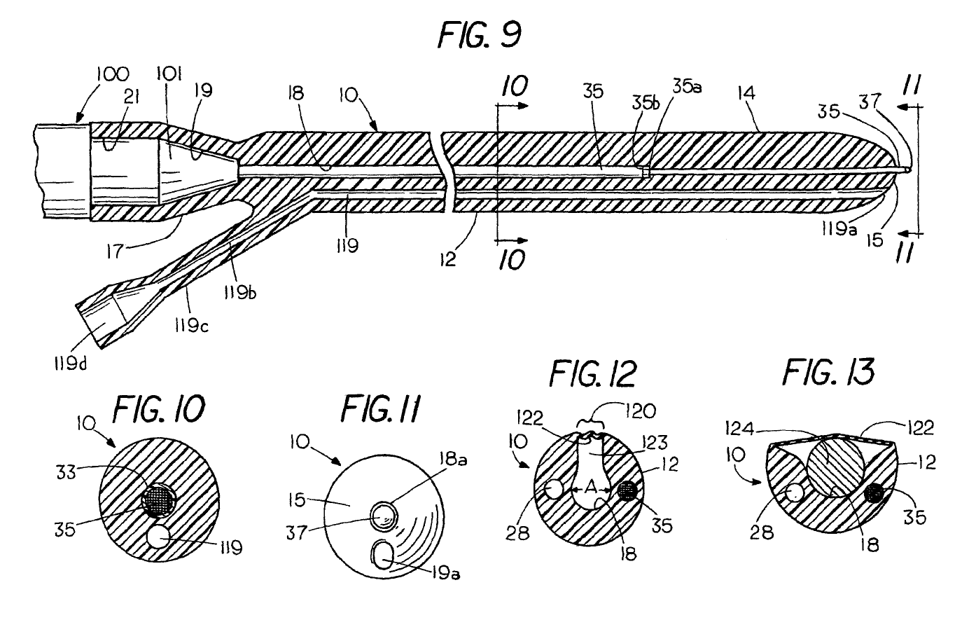

Refer now to FIGS. 8-11, which illustrate a visually directed nasogastric tube in accordance with the invention wherein the same numerals refer to corresponding parts already described. In this embodiment, the flexible optical cable 35 may be connected at 100 to a viewing instrument 83. In this embodiment, the cable 35 includes a tapered barrel 101 that fits into a tapered socket 19 within the catheter 12. As described earlier, a light source may be provided to which the optic cable 35 may be connected. In FIG. 8, a light source and camera assembly (not shown) similar to 82 of FIGS. 1 and 14 may be provided within monitor 83 for directing light into the fiber-optic bundle 35 and out through the lens 37 to illuminate the field just ahead of the tip 15 of the instrument 10. The image proximate the lens 37 may be then carried back through the fiber-optic bundle 35 to the monitor 83 and viewing screen 84. Cable 35 extends from port 21 at the proximal end of the channel 18 to the distal end 14 and as shown in FIG. 9 for most purposes projects slightly beyond the tip 15 of the catheter 12. With the objective viewing lens 37 located just beyond the tip 15 of the catheter, enhanced viewing ahead and also to the side may be made possible by the wide angle of view that is permitted both while the catheter 12 may be being inserted as well as after it is in place within the body of the patient. In the nasogastric tube of FIGS. 8-11, the fiber-optic bundle 35 may be preferably removable.

As shown in FIG. 9, the cable 35 may have a distal segment of reduced diameter which can be any length, e.g. 2-3 inches long to define a shoulder 35a in the cable so as to provide a proximal portion having a relatively large diameter and a distal segment of a reduced diameter with a shoulder between them which acts as a retainer. The channel 18 in the catheter may be shaped like the cable 35. Thus, when the cable is fully inserted, the shoulder 35a rests against a similarly shaped restriction in the channel 18 which serves as a retainer or stop to check the distal movement of the cable. In a preferred form, a circular washer 35b of a selected thickness and having an outside diameter the same as the larger diameter of the cable 35 may be mounted on the cable at the shoulder 35a to act as a retainer for determining the position of lens 37 relative to the tip 15 of the catheter 12 during use to thereby control the extension, if any, of lens 37 beyond the tip 15.

FIG. 9 thus shows a removable transmission cable 35 slideably mounted within a channel 18 as well as a working channel 119 positioned laterally of the channel 18. Channel 119 may have an outlet port 119a at the distal tip of the instrument just below the outlet port through which the cable 35 extends. The proximal end of the working channel 119 extends at 119b through a proximal extension 119c terminating at an opening 119d through which fluid can be drained from the body or surgical instruments can be passed when required through the catheter 12 into the patient. The fibers within the cable 35 can be enclosed within a tubular casing 33 (FIG. 10) to hold the fibers together. During use, as shown in FIG. 8, the visually directed instrument 10 comprising a nasogastric tube can be held in place conventionally where it enters the nose with adhesive tape (not shown) and accordingly no balloon may be required for holding the tube in place or within the body. The viewing instrument 100 as shown in FIG. 8 may be connected by means of a cable 35 to the visual display 83 which includes the video display screen 87 for continuously displaying in real time an image of the area just ahead of the distal tip 15 of the instrument.

Instrument 10 comprising the visually directed nasogastric tube may be used for patients who are unable to ingest nutrients by mouth and may be inserted through either nostril and passed down through the pharynx and esophagus into the stomach, typically for short-term feeding. Placement must be checked before each feeding. This can be done by viewing the area just ahead of the tip 15 by displaying it on the viewing screen 87. Another use for the nasogastric tube is to drain accumulated fluids from the stomach and small intestine due to a blockage of the bowel from an obstruction or bowel inactivity. The present invention may be particularly advantageous in overcoming the problems that resulted previously from the conventional feeding tube curling up in the esophagus, becoming diverted into the trachea, or coming to rest in a less than optimal location in the stomach. When these problems arose prior to the present invention, the solution was to take a static x-ray (using abdominal film) or measure the presence of CO₂ to rule out placement of the tube in the trachea. These procedures were complicated and took time since it was necessary to move the patient to the radiology department or transport x-ray equipment to the patient's room for the x-rays, adjust the tube, then take additional x-rays to verify the actual location of the tube and, of course, a radiologist may be required to read the x-rays.

The visually directed nasogastric tube in accordance with the invention thus may have two lumens; channel 18 in which the visual element or cable 35 may be preferably removably mounted and the working channel 119, which serves as the primary working channel for drainage and/or feeding. However, if the visual element 35 is removed, channel 18 can also be used as a working channel, for example, to pass an instrument or succession of instruments through the catheter 12 into the body of the patient. Consequently, the invention provides continuous visually directed insertion of the catheter while also providing, if desired, a pair of parallel laterally spaced apart working channels that can each be used as a working channel for different purposes during surgery or convalescence. For example, channel 18 can be used for drainage while at the same time the channel 119 may be used for inserting and removing a variety of surgical instruments or guide wires through the catheter which then acts as a protective sheath that reduces discomfort, eliminates pain that would otherwise be experienced, and the tissue trauma that would occur if the instruments were passed directly through a body opening without the catheter 12 in place. Channel 18 which may be preferably the largest in diameter is well suited for drainage and/or feeding the patient. When the visual element 35 is removable, it may be preferably enclosed within the flexible protective plastic casing 33 and coated on the outside with a suitable surgical lubricant so that it can be removed when desired from the instrument 10. The visual element 35 and casing 33 also provides a degree of stiffness for the catheter 12 so that it can be reliably pushed through a tight passage and yet may be able to flex freely around and through curved body openings and easily pass obstructions. In such a case, the visual element acts to assist in insertion and thus serves as an obturator for adding a degree of stiffness to the catheter.

It will be thus understood that the invention provides continuous visually directed placement as well as allowing the position of the distal end of the instrument to be confirmed by the operator at the time of placement. Consequently, it eliminates the need for x-rays and the services of a radiologist to read them as well as the need for a CO₂ determination procedure. As already described in connection with FIGS. 1-7, in place of a visual sensor, the invention can employ any other known form of sensor for evaluating one or more conditions along the length of the cable 35 or in the region just ahead of the tip 15 of the instrument, e.g. a chemical sensor, a temperature sensor, a pressure sensor, etc.

To more fully explain the invention and the results that can be achieved, an additional example will be presented to illustrate its capabilities. Once the instrument 10 comprising the nasogastric tube (FIGS. 8-11) is in place within the stomach, the visual element 35 and the light beam on axis 39 provided by the light source 84 permits the doctor to identify the exact location for retrograde placement of a percutaneous guide wire, that is to say, where a hole is to be punched with the guide wire from the outside of the patient through the skin of the abdomen into the stomach while being guided by the light within the stomach that may be directed as a beam through the lens 37. The light transmitted along the optic axis 39 (FIG. 8) at the tip 15 of the instrument 10 comprising the nasogastric tube may be bright enough for the doctors to see it by transillumination through the skin when observing the patient from the exterior. The beam can be positioned conventionally by guide wires (not shown) in the catheter 12 as described in U.S. Pat. No. 6,994,667. The doctor can then choose to insert the guide wire from the inside out (antegrade) through the lateral working channel 119 while the light is on, or from the outside in retrograde, whichever is preferred. If the retrograde procedure is used, the guide wire may be inserted from the exterior of the body through the skin into the stomach at the exact location of the light transmitted from the tip 15 of the instrument along the axis 39 (FIG. 8). Thus, the visual element of 35 of the instrument 10 comprising the nasogastric tube allows the doctor to place the guide wire precisely. The instrument 10 comprising the nasogastric tube may be then used as a working channel device to pull the guide wire and/or feeding tube of FIG. 7 into the stomach via the working channel 119. On the other hand, in the antegrade procedure, the doctor is assisted by the light from the visual element to correctly pass the guide wire from the stomach out through the skin of the abdomen.

Refer now to FIGS. 12 and 13 which illustrate a modified form of the invention in which the catheter 12 may be provided with a longitudinally extending area designated 120 running throughout the length of the catheter that may have a reduced wall thickness which may be bridged across by a stretchy elastic sheet or band 122. The reduced wall thickness can be seen in FIG. 12 as a gap 123 adjacent band 122. During use, when the catheter 12 is in a relaxed resting state as shown in FIG. 12, the lumen 18 may have a predetermined diameter A capable of accommodating surgical instruments of a certain size that are to be passed through it. However, as shown in FIG. 13, when a surgical instrument 124 of a much larger size may be passed through the lumen 18, the elastic band 122 that covers the area of reduced wall thickness, the band 122 becomes stretched as the wall of the catheter 12 may be extended by the instrument 124, thus allowing surgical instruments 124 of a much larger size than the initial diameter of lumen 18 to be passed through the catheter 12 and into the body of the patient for carrying out various surgical procedures, e.g. cauterization, tumor removal, or for other purposes. The invention thus provides an expansion zone 120 extending the length of the catheter 12 that may be bridged by the relatively thin elastic expansion band 122 so as to allow enlargement of the lumen 18 along the entire length of the catheter 12 for introducing or removing instruments 124 that are larger than the lumen 18.

The band 122 over the thin wall area at 120 thus provides a catheter having a greatly expandable lumen 18 yet which maintains its integrity, i.e. lumen 18 does not open out into the body passage or communicate with any other part of the body except through the opening at the distal tip 15. The catheter is therefore able to expand substantially to enable oversize instruments such as that shown at 124 to be passed into the body, yet the wall of the body opening may be protected at all times by the catheter and the elastic band 122 so as to avoid injury that might otherwise be induced by the instrument 124 as it is being inserted or retracted.

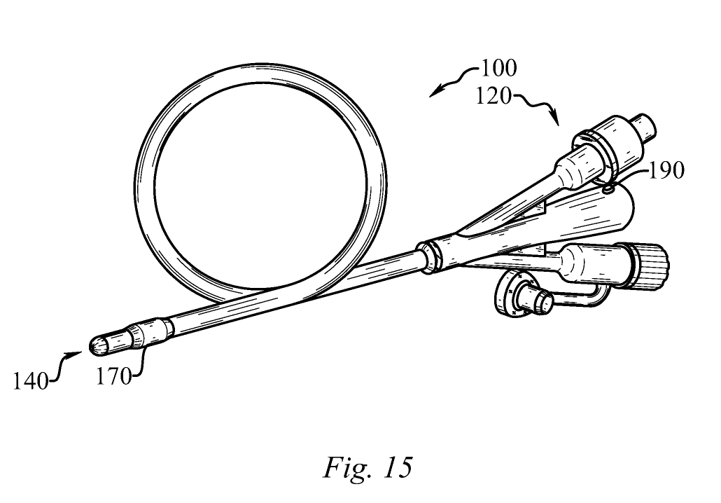

In yet other embodiments, seen well if FIGS. 15 through 29, as with the embodiments detailed above, of the flexible visually directed medical intubation instrument and method, a medical intubation instrument (100) enables a significant advance in the state of the art. The preferred embodiments of the instrument (100) accomplish this by new and novel arrangements of elements and methods that are configured in unique and novel ways and which demonstrate previously unavailable but preferred and desirable capabilities. The description set forth below in connection with the drawings is intended merely as a description of the presently preferred embodiments of the instrument (100), and is not intended to represent the only form in which the instrument (100) may be constructed or utilized. The description sets forth the designs, functions, means, and methods of implementing the instrument (100) in connection with the illustrated embodiments. It is to be understood, however, that the same or equivalent functions and features may be accomplished by different embodiments that are also intended to be encompassed within the spirit and scope of the claimed instrument (100) and method.

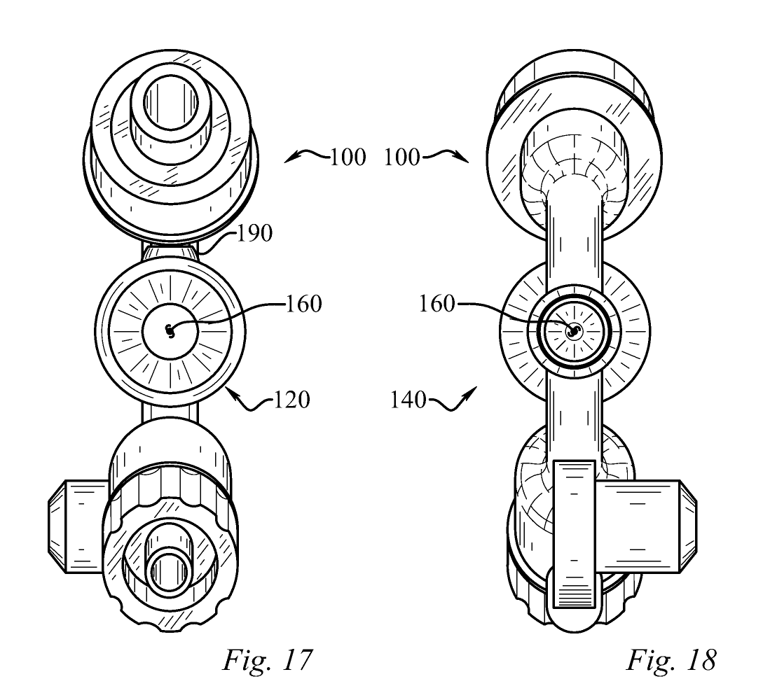

A medical intubation instrument may include a catheter (100) formed of a flexible biocompatible polymeric material having a catheter proximal end (120) and a catheter distal end (140), as seen in FIGS. 15-18. The catheter (100) may, in some embodiments, and as seen in FIGS. 15-17, may have a catheter external orientation mark (190) to indicate any directionality of the catheter distal end (140), which, of course, cannot be seen by the clinician after entry to the body. Referring now to FIGS. 17-19, 25, and 29, the catheter proximal end (120) and the catheter distal end (140) may be in fluid communication through at least one catheter longitudinal channel (160) extending from the catheter proximal end (120) to the catheter distal end (140).

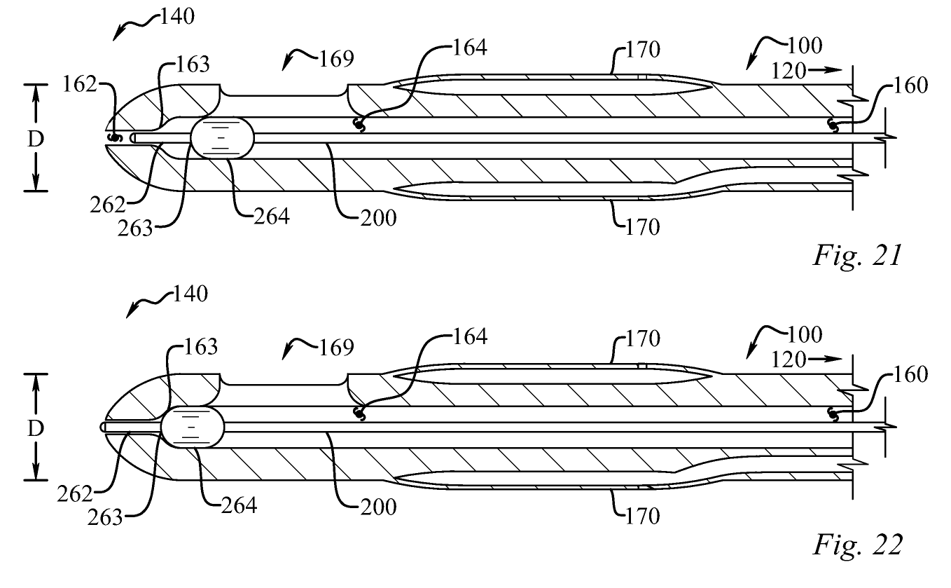

As seen by way of example in FIGS. 21-22, within the catheter (100), there may be at least a catheter longitudinal channel first diameter portion (162) and a catheter longitudinal channel second diameter portion (164) greater in diameter than the catheter longitudinal channel first diameter portion (162). The catheter longitudinal channel first diameter portion (162) and the catheter longitudinal channel second diameter portion (164) are in fluid communication in at least one catheter longitudinal channel diameter transition area (163), again as seen in FIGS. 21-22.

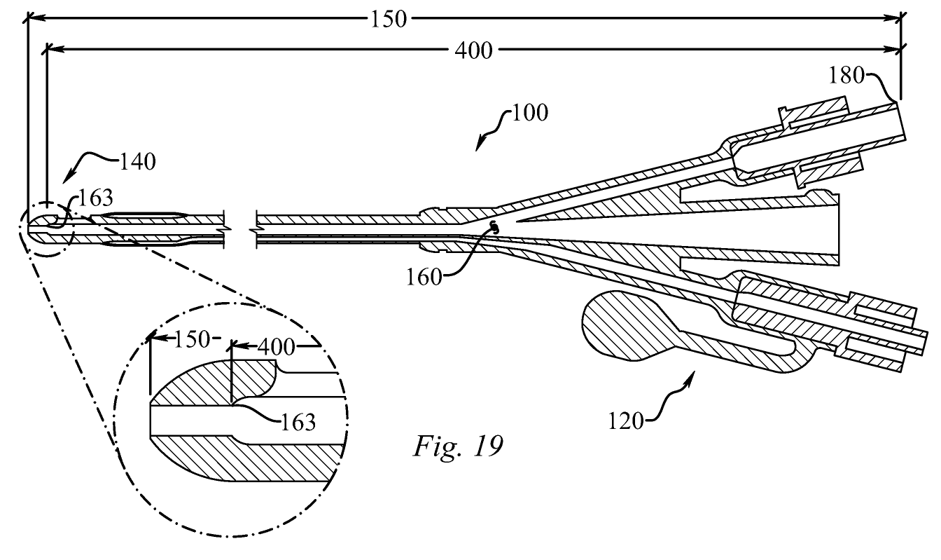

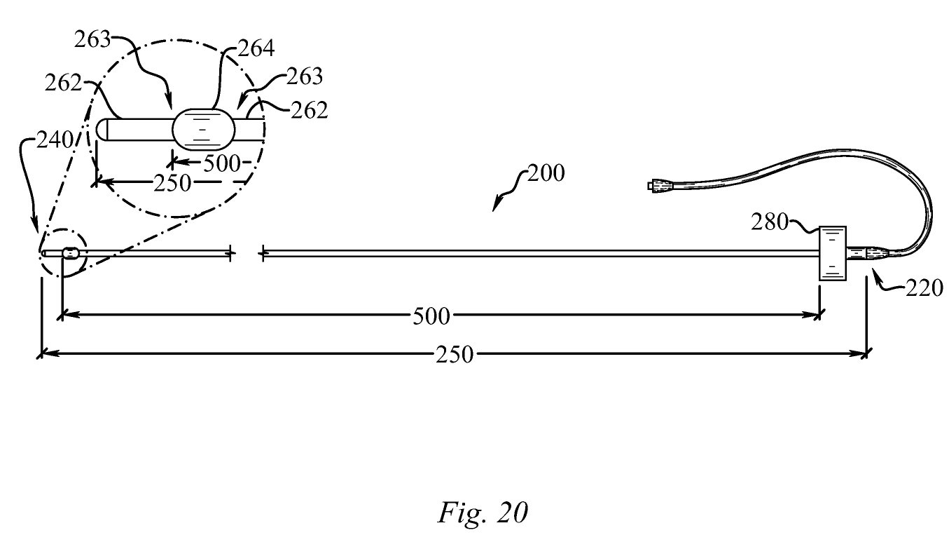

As seen well in FIGS. 20-22, another feature includes at least one sensor (200) having a sensor proximal end (220) and a sensor distal end (240) having at least a sensor first diameter portion (262) and at least a sensor second diameter portion (264) greater in diameter than the sensor first diameter portion (262). The at least a sensor first diameter portion (262) and at least a sensor second diameter portion (264) are connected by an at least one intermediary sensor diameter transition portion (263), seen well in FIGS. 21-22, and at least a portion of the sensor (200) may be releasably contained within the at least one catheter longitudinal channel (160) during medical intubation of a living subject. The catheter longitudinal channel diameter transition area (163) and the at least one sensor diameter transition portion (263) cooperate to reversibly position the catheter (100) and the sensor (200) in a predetermined spatial relationship between a catheter axial length (150) and a sensor axial length (250) during intubation, as seen in FIGS. 19-20. A typical relationship as seen during intubation is seen by way of example in FIG. 22, while FIG. 21 shows a typical relationship as a sensor (200) is being slightly withdrawn from a catheter (100) following intubation.

While no particular number of catheter longitudinal channels (160) are envisioned, these being limited primarily by dictates of size, one skilled in the art will appreciate an embodiment where the catheter (100) may have a single catheter longitudinal channel (160), thus maximizing the possible diameter of the single catheter longitudinal channel (160) relative to an overall catheter diameter (D), as seen by way of example and not limitation in FIGS. 21-22.

While it is possible for a sensor to take up all, or most, of the lumen of any given catheter longitudinal channel (160), it is also envisioned that the sensor (200) may take up only a relatively small amount of the catheter longitudinal channels (160). Embodiments are specifically envisioned where a diameter of the catheter longitudinal channel second diameter portion (164) exceeds a diameter of the sensor first diameter portion (262) by a ratio of at least two to one, three to one, and four to one.

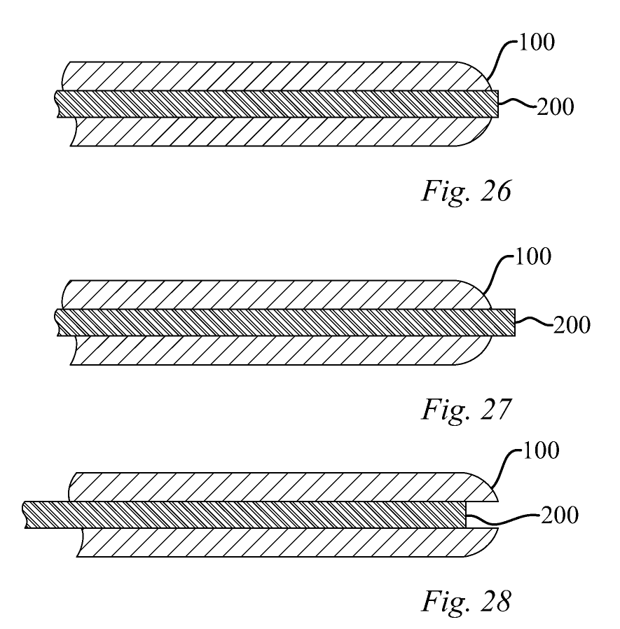

Relative motion between catheters (100) and sensors (200) they contain can create a problem that is hypothetically illustrated in FIGS. 26-28. In many applications, it may be desirable for the tip of a sensor (200), to lie flush with the end of the catheter (100), or to perhaps extend from the tip of the catheter (100) by a very small amount, as seen in FIG. 26. This optimizes the field of view of a sensor (200), particularly an optical sensor (200). At the same time, it is generally undesirable for a sensor to extend too far beyond the tip of a catheter (100), such as illustrated in FIG. 27, lest it injure delicate biological tissues. This is particularly true when the nature of the sensor (200), for example, if the sensor (200) were a fiber-optic bundle, is relatively rigid.

Many catheters (100) as envisioned by the instant invention are made of somewhat soft and stretchable materials. As a result, during the pushing and pulling of intubation and removal, the catheter (100) is apt to be both compressed (made shorter) and stretched (made longer). One skilled in the art will readily see that if a relatively rigid sensor (200) lies within a relatively stretchable catheter (100) that is compressed in length, and all other things are equal, the tip of the sensor (200) will tend to extend from the tip of the catheter (100), as seen in FIG. 27. This creates a potentially hazardous situation, as described above.

However, if the distance between the catheter distal end (140) and the catheter longitudinal channel diameter transition area (163) is relatively short, this effect is minimized, as the interaction between the catheter longitudinal channel first diameter portion (162) and a catheter longitudinal channel second diameter portion (164) creates a “stop” that prevents the sensor (200) from unduly extending beyond the tip of the catheter (100).

However, in such a case, the distance between the catheter proximal end (120) and the catheter longitudinal channel diameter transition area (163) is likely to be relatively long, and therefore a reciprocal problem is created if the catheter (100) is not compressed, but rather is stretched. If the catheter (100) is stretched, the situation may develop as seen in FIG. 28, where the sensor (200) tends to be relatively drawn back down the catheter longitudinal channel (160). This tends to restrict the field of view of the sensor (200), much as if a viewer tried to look through a length of pipe.

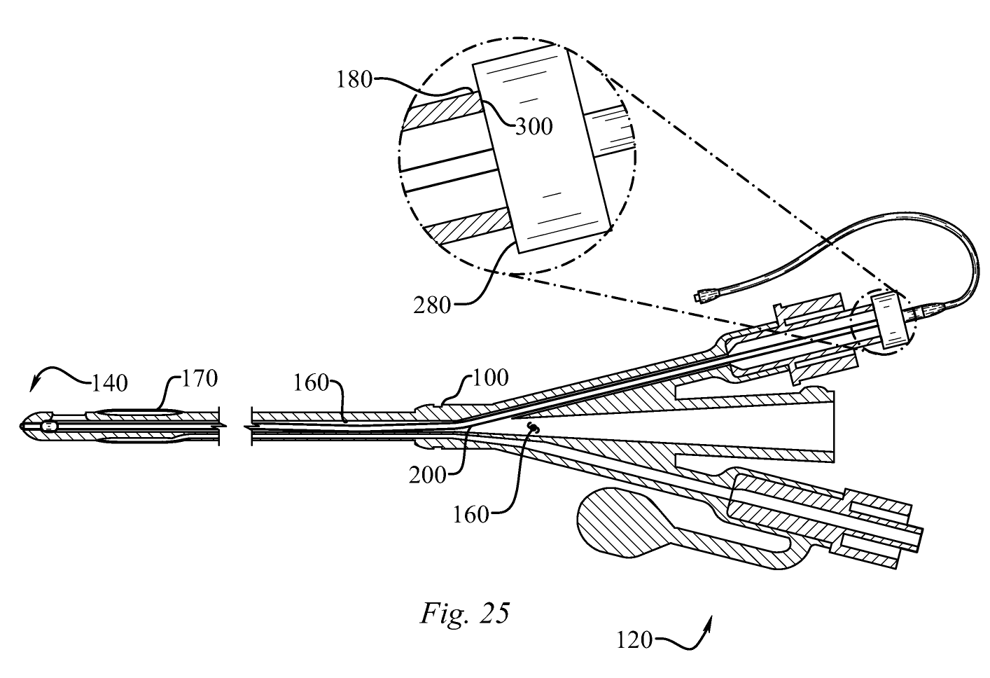

To minimize such an effect, an embodiment, such as is well seen in FIG. 25, is envisioned where the catheter proximal end (120) and the sensor proximal end (220) cooperate to create a releasable catheter-sensor fixation point (300) at a predetermined distance along the catheter longitudinal channel (160) from the catheter longitudinal channel diameter transition area (163). Thus, a length relationship between the catheter (100) and the sensor (200) tends to be established near both the catheter proximal end (120) and the catheter distal end (140).

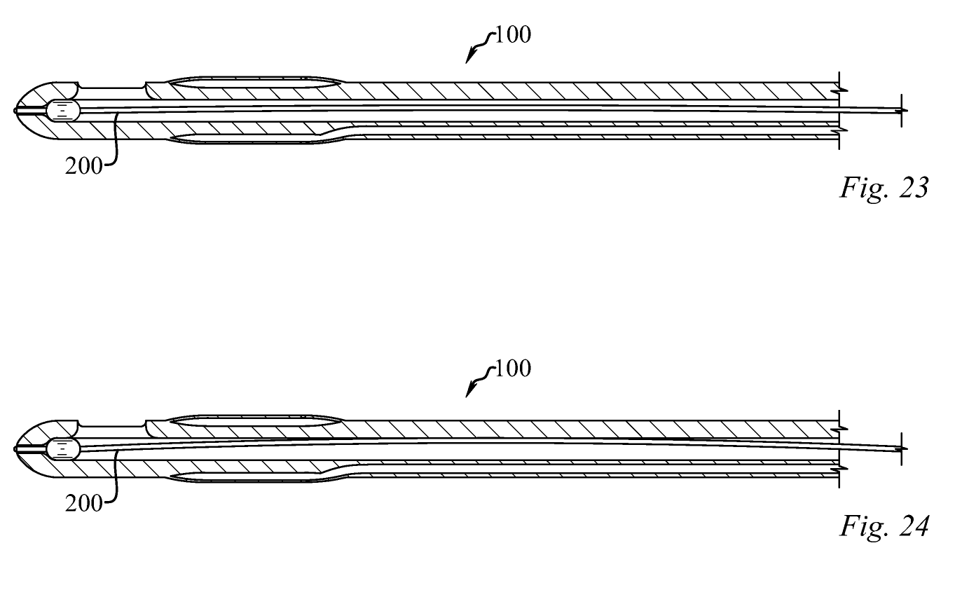

Thus, it may be said that the catheter (100) may have a catheter working length (400), seen well in FIG. 19, between the catheter-sensor fixation point (300), seen well in FIG. 25, and the catheter longitudinal channel diameter transition area (163), seen well in FIG. 22. The sensor (200), seen well in FIG. 20, may have a sensor working length (500), seen well in FIG. 20 between catheter-sensor fixation point (300), seen well in FIG. 25, and the at least one sensor diameter transition portion (263), seen again in FIG. 20. If the sensor working length (500) is made slightly longer than the catheter working length (400), the extra length will tend to accumulate within the catheter longitudinal channel (160), seen as a slight bow in the sensor (200) in FIG. 24. The magnitude of such a bow will be readily ascertainable by one skilled in the art, and will serve as a reservoir of length in the case of compression or stretching of the catheter (100). If the catheter (100) is compressed, extra sensor (200) length will tend to increase the bow. If the catheter (100) is stretched, the bow will tend to straighten, as seen in FIG. 23. In either case, an optimal relationship between the sensor (200) and the catheter proximal end (120) will tend to be preserved. Embodiments are envisioned wherein the sensor working length (500) is at least two percent, five percent, and ten percent longer than the catheter working length (400).

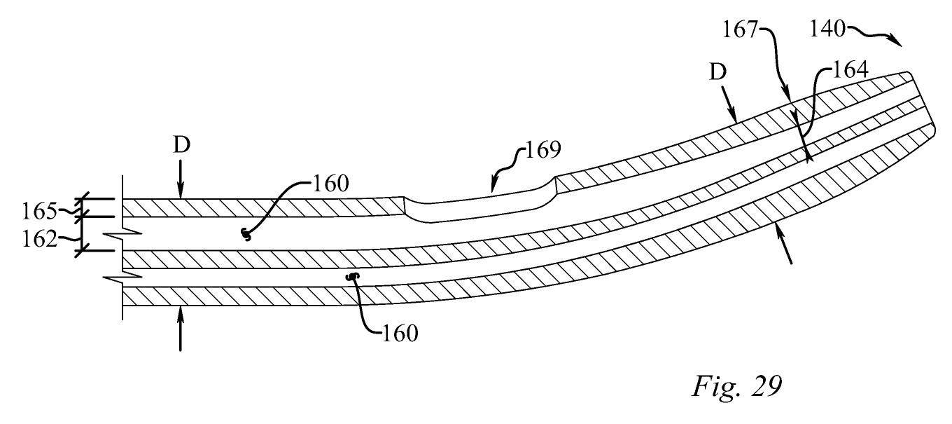

In another embodiment, seen in FIG. 29, the catheter longitudinal channel first diameter portion (162) cooperates with a catheter longitudinal channel first diameter portion wall thickness (165) and the catheter longitudinal channel second diameter portion (164) cooperates with a catheter longitudinal channel second diameter portion wall thickness (167) to form at least one uniform external catheter diameter (D). This uniform diameter (D) extends over at least a portion of the catheter longitudinal channel first diameter portion (162) and at least a portion of the catheter longitudinal channel second diameter portion (164). In such an embodiment, the distal end (140) of the catheter (100) may have a slightly thickened wall diameter, without increasing the overall diameter of the catheter, which tends to promote stiffness and prevent kinking near the catheter distal end (140). In this, as in all other embodiments envisioned in this teaching, the catheter (100) may have a straight distal end (140), a slightly curved distal end (140), or any other configuration which would be known to one skilled in the art.

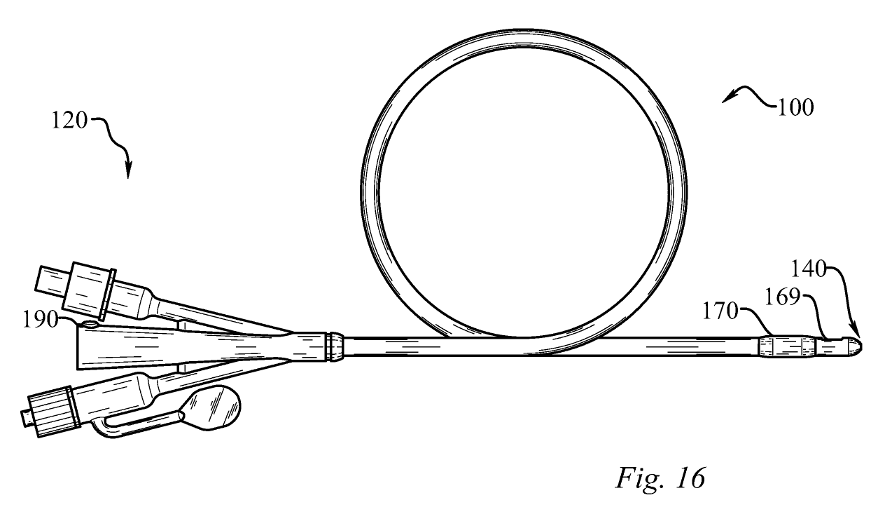

In yet other embodiments, the at least one catheter longitudinal channel (160) is in fluid communication with an ambient atmosphere at a catheter longitudinal channel side port (169), such as is seen well in FIGS. 16, 21-22, and 29, intermediate between the catheter proximal end (120) and the catheter distal end (140) and distal to an inflatable retention balloon (170). Thus, such a catheter longitudinal channel side port (169) may allow free flow of fluid into the catheter longitudinal channel (160) even if the sensor (200) occupies all or substantially all of the catheter longitudinal channel first diameter portion (162), as seen in FIGS. 21-22.

In such an embodiment, as seen well in FIGS. 21-22, the catheter (100) may have only a single catheter longitudinal channel (160), thus maximizing the potential diameter of the catheter longitudinal channel (160) relative to a given catheter diameter (D). Particularly if the sensor first diameter portion (262) may have a relatively small diameter compared with the catheter longitudinal channel second diameter portion (164), flow through the catheter longitudinal channel second diameter portion (164) may be only minimally diminished. Embodiments are envisioned in which the catheter longitudinal channel (160) thereby remains patent to an overall flow rate of a liquid during intubation with a sensor (200) in place of at least 80% of a flow rate of the same liquid through the catheter longitudinal channel (160) with the sensor (200) removed from the one catheter longitudinal channel (160). In particular, embodiments are envisioned where a diameter of the catheter longitudinal channel second diameter portion (164) exceeds a diameter of the sensor first diameter portion (262) by a ratio of at least two to one, three to one, and four to one.

One skilled in the art will readily see that the devices and elements detailed above lend themselves to a method of direct vision medical intubation of a living subject. Such a method would include the steps of placing at least one sensor (200), according to the teaching detailed above, at least partially within a catheter (100) according to the teachings above, and intubating a living subject with the catheter (100) and sensor (200) under direct observation with the sensor (200). During such intubation, it is envisioned that the at least one catheter longitudinal channel (160) will remain patent to an overall flow rate of a liquid during intubation with a sensor (200) in place of at least 80% of a flow rate of the same liquid through the at least one catheter longitudinal channel (160) with the sensor (200) removed from the one catheter longitudinal channel (160).

Numerous alterations, modifications, and variations of the preferred embodiments disclosed herein will be apparent to those skilled in the art and they are all anticipated and contemplated to be within the spirit and scope of the instrument (100). For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the instrument (100) are described herein, it is to be understood that the practice of such additional modifications and variations and the equivalents thereof, are within the spirit and scope of the instrument (100) as defined in the following claims.

What is claimed is:

1. A medical intubation instrument comprising,
a catheter (100) formed of a flexible biocompatible polymeric material having a catheter proximal end (120) and a catheter distal end (140), wherein the catheter proximal end (120) and the catheter distal end (140) are in fluid communication through at least one catheter longitudinal channel (160) extending from the catheter proximal end (120) to the catheter distal end (140), the at least one catheter longitudinal channel (160) having at least a catheter longitudinal channel first diameter portion (162) and a catheter longitudinal channel second diameter portion (164) greater in diameter than the catheter longitudinal channel first diameter portion (162), and the catheter longitudinal channel first diameter portion (162) and the catheter longitudinal channel second diameter portion (164) are in fluid communication in at least one catheter longitudinal channel diameter transition area (163);
at least one sensor (200) having a sensor proximal end (220) and a sensor distal end (240), the at least one sensor (200) having at least a sensor first diameter portion (262) and at least a sensor second diameter portion (264) greater in diameter than the sensor first diameter portion (262), wherein the at least a sensor first diameter portion (262) and at least a sensor second diameter portion (264) are connected by an at least one intermediary sensor diameter transition portion (263), and
wherein at least a portion of the at least one sensor (200) is releasably contained within the at least one catheter longitudinal channel (160) during medical intubation of a living subject, and the at least one catheter longitudinal channel diameter transition area (163) and the at least one intermediary sensor diameter transition portion (263) cooperate to reversibly position the catheter (100) and the at least one sensor (200) in a predetermined spatial relationship between a catheter axial length (150) and a sensor axial length (250) during intubation.

2. The device according to claim 1, wherein the catheter (100) has a single catheter longitudinal channel (160).

3. The device according to claim 1, wherein a diameter of the catheter longitudinal channel second diameter portion (164) exceeds a diameter of the sensor first diameter portion (262) by a ratio of at least two to one.

4. The device according to claim 1, wherein a diameter of the catheter longitudinal channel second diameter portion (164) exceeds a diameter of the sensor first diameter portion (262) by a ratio of at least three to one.

5. The device according to claim 1, wherein a diameter of the catheter longitudinal channel second diameter portion (164) exceeds a diameter of the sensor first diameter portion (262) by a ratio of at least four to one.

6. The device according to claim 1, wherein the catheter proximal end (120) and the sensor proximal end (220) cooperate to create a releasable catheter-sensor fixation point (300) at a predetermined distance along the at least one catheter longitudinal channel (160) from the at least one catheter longitudinal channel diameter transition area (163).

7. The device according to claim 6, wherein the catheter (100) has a catheter working length (400) between the catheter-sensor fixation point (300) and the at least one catheter longitudinal channel diameter transition area (163), and the at least one sensor (200) has a sensor working length (500) between the catheter-sensor fixation point (300) and the at least one intermediary sensor diameter transition portion (263), wherein the sensor working length (500) is at least two percent longer than the catheter working length (400).

8. The device according to claim 6, wherein the catheter (100) has a catheter working length (400) between the catheter-sensor fixation point (300) and the at least one catheter longitudinal channel diameter transition area (163), and the at least one sensor (200) has a sensor working length (500) between the catheter-sensor fixation point (300) and the at least one intermediary sensor diameter transition portion (263), wherein the sensor working length (500) is at least five percent longer than the catheter working length (400).

9. The device according to claim 6, wherein the catheter (100) has a catheter working length (400) between the catheter-sensor fixation point (300) and the at least one catheter longitudinal channel diameter transition area (163), and the at least one sensor (200) has a sensor working length (500) between the catheter-sensor fixation point (300) and the at least one intermediary sensor diameter transition portion (263), wherein the sensor working length (500) is at least ten percent longer than the catheter working length (400).

10. The device according to claim 1, wherein the catheter longitudinal channel first diameter portion (162) cooperates with a catheter longitudinal channel first diameter portion wall thickness (165) and the catheter longitudinal channel second diameter portion (164) cooperates with a catheter longitudinal channel second diameter portion wall thickness (167) to form at least one uniform external catheter diameter (D) over at least a portion of the catheter longitudinal channel first diameter portion (162) and at least a portion of the catheter longitudinal channel second diameter portion (164).

11. The device according to claim 1, wherein the at least one catheter longitudinal channel (160) is in fluid communication with an ambient atmosphere at a catheter longitudinal channel side port (169) intermediate between the catheter proximal end (120) and the catheter distal end (140) and distal to an inflatable retention balloon (170).

12. A medical intubation instrument comprising,
a catheter (100) formed of a flexible biocompatible polymeric material having a catheter proximal end (120) and a catheter distal end (140), wherein the catheter proximal end (120) and the catheter distal end (140) are in fluid communication through one catheter longitudinal channel (160) extending from the catheter proximal end (120) to the catheter distal end (140), the one catheter longitudinal channel (160) having at least a catheter longitudinal channel first diameter portion (162) and a catheter longitudinal channel second diameter portion (164) greater in diameter than the catheter longitudinal channel first diameter portion (162), and the catheter longitudinal channel first diameter portion (162) and the catheter longitudinal channel second diameter portion (164) are in fluid communication in at least one catheter longitudinal channel diameter transition area (163);
at least one sensor (200) having a sensor proximal end (220) and a sensor distal end (240), the at least one sensor (200) having at least a sensor first diameter portion (262) and at least a sensor second diameter portion (264) greater in diameter than the sensor first diameter portion (262), wherein the at least a sensor first diameter portion (262) and at least a sensor second diameter portion (264) are connected by at least one intermediary sensor diameter transition portion (263),
wherein at least a portion of the at least one sensor (200) is releasably contained within the one catheter longitudinal channel (160) during medical intubation of a living subject, and the at least one catheter longitudinal channel diameter transition area (163) and the at least one intermediary sensor diameter transition portion (263) cooperate to reversibly position the catheter (100) and the at least one sensor (200) in a predetermined spatial relationship between a catheter axial length (150) and a sensor axial length (250) during intubation, and
wherein the one catheter longitudinal channel (160) remains patent to an overall flow rate of a liquid during intubation with the at least one sensor (200) in place of at least 80% of a flow rate of the same liquid through the one catheter longitudinal channel (160) with the at least one sensor (200) removed from the one catheter longitudinal channel (160).

13. The device according to claim 12, wherein a diameter of the catheter longitudinal channel second diameter portion (164) exceeds a diameter of the sensor first diameter portion (262) by a ratio of at least two to one.

14. The device according to claim 12, wherein a diameter of the catheter longitudinal channel second diameter portion (164) exceeds a diameter of the sensor first diameter portion (262) by a ratio of at least three to one.

15. The device according to claim 12, wherein a diameter of the catheter longitudinal channel second diameter portion (164) exceeds a diameter of the sensor first diameter portion (262) by a ratio of at least four to one.

16. The device according to claim 12, wherein the catheter proximal end (120) and the sensor proximal end (220) cooperate to create a releasable catheter-sensor fixation point (300) at a predetermined distance along the one catheter longitudinal channel (160) from the at least one catheter longitudinal channel diameter transition area (163).

17. The device according to claim 16, wherein the catheter (100) has a catheter working length (400) between the catheter-sensor fixation point (300) and the at least one catheter longitudinal channel diameter transition area (163), and the at least one sensor (200) has a sensor working length (500) between the catheter-sensor fixation point (300) and the at least one intermediary sensor diameter transition portion (263), wherein the sensor working length (500) is at least two percent longer than the catheter working length (400).

18. The device according to claim 16, wherein the catheter (100) has a catheter working length (400) between the catheter-sensor fixation point (300) and the at least one catheter longitudinal channel diameter transition area (163), and the at least one sensor (200) has a sensor working length (500) between catheter-sensor fixation point (300) and the at least one intermediary sensor diameter transition portion (263), wherein the sensor working length (500) is at least five percent longer than the catheter working length (400).

19. The device according to claim 16, wherein the catheter (100) has a catheter working length (400) between the catheter-sensor fixation point (300) and the at least one catheter longitudinal channel diameter transition area (163), and the at least one sensor (200) has a sensor working length (500) between the catheter-sensor fixation point (300) and the at least one intermediary sensor diameter transition portion (263), wherein the sensor working length (500) is at least ten percent longer than the catheter working length (400).

20. A method of direct vision medical intubation of a living subject, comprising the steps of:
a. placing at least one sensor (200) having a sensor proximal end (220) and a sensor distal end (240) at least partially within a catheter (100) formed of a flexible biocompatible polymeric material having a catheter proximal end (120) and a catheter distal end (140), wherein the catheter proximal end (120) and the catheter distal end (140) are in fluid communication through at least one catheter longitudinal channel (160) extending from the catheter proximal end (120) to the catheter distal end (140), such that the catheter (100) and the at least one sensor (200) are in a predetermined spatial relationship between a catheter axial length (150) and a sensor axial length (250) during intubation, and
b. intubating a living subject with the catheter (100) and at least one sensor (200) under direct observation with the at least one sensor (200) while maintaining the at least one catheter longitudinal channel (160) patent to an overall flow rate of a liquid during intubation with the at least one sensor (200) in place of at least 80% of a flow rate of the same liquid through the at least one catheter longitudinal channel (160) with the at least one sensor (200) removed from the at least one catheter longitudinal channel (160).
